(19) United States
(12) Patent Application Publication
(10) Pub. No.: US 2023/0172922 A1
(43) Pub. Date: Jun. 8, 2023

(54) METHODS OF TREATMENT OF TRIGEMINAL NEURALGIA

(71) Applicant: Noema Pharma AG, Basel (CH)

(72) Inventor: George Garibaldi, Basel (CH)

(21) Appl. No.: 18/102,532

(22) Filed: Jan. 27, 2023

Related U.S. Application Data
(63) Continuation of application No. PCT/EP2021/071376, filed on Jul. 30, 2021.
(60) Provisional application No. 63/058,630, filed on Jul. 30, 2020.

(51) Int. Cl. A61K 31/4439

(52) U.S. Cl. CPC A61K 31/4439

(57) ABSTRACT

Provided herein are methods of treating trigeminal neuralgia in a subject in need thereof by administering to the subject compositions comprising an mGlu5 negative allosteric modulator (NAM), having the structure of Formula I:

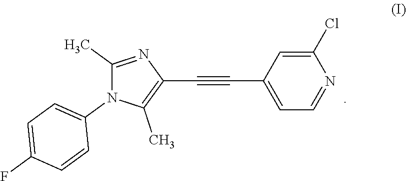 [SMILES: Cc1nc(C#Cc2ccnc(Cl)c2)c(C)n1-c1ccc(F)cc1]

### CROSS-REFERENCE TO RELATED APPLICATION

This application is a continuation of International Application Number PCT/EP2021/071376 filed on Jul. 30, 2021, which claims priority to U.S. Ser. No. 63/058,630 filed on Jul. 30, 2020, the contents of which is incorporated herein by reference in its entirety.

### FIELD

The present disclosure relates to the field of medicine and the treatment of trigeminal neuralgia. More specifically, the present disclosure relates to use of compositions comprising 2-chloro-4-[1-(4-fluorophenyl)-2,5-dimethyl-1H-imidazol-4-ylethynyl]pyridine, or a pharmaceutically acceptable salt thereof, in the treatment or amelioration of trigeminal neuralgia.

### BACKGROUND

Trigeminal neuralgia (TGN or TN) is a chronic pain condition that affects the trigeminal nerve, which carries sensation from the face to brain. TGN includes typical and atypical trigeminal neuralgia, as well as classical, secondary and idiopathic TGN. The typical form results in episodes of severe, sudden, shock-like pain in one side of the face that lasts for seconds to a few minutes, while the atypical form results in a constant burning pain that is less severe. The pain resulted from TGN has a significant impact on activities of daily living, which can lead to severe depression and anxiety.

While medications such as anticonvulsants (e.g., carbamazepine) or antidepressants (e.g., amitriptyline) can help alleviate the pain caused by TGN, some of these medications have significant side effects, thereby limiting their medical use.

Therefore, there is an unmet medical need to develop new methods for treating TGN without significant side effects.

### SUMMARY

In one aspect, provided herein are methods of treating trigeminal neuralgia (TGN), comprising administering to a subject in need thereof a composition comprising a therapeutically effective amount of a compound or a pharmaceutically acceptable salt thereof, wherein the compound is of Formula I:

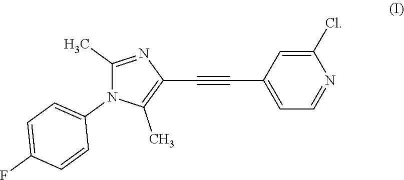 [SMILES: Cc1nc(C#Cc2ccnc(Cl)c2)c(C)n1-c1ccc(F)cc1]

In some embodiments, treating uses a composition comprising a crystalline anhydrate form (Form A) of a monosulfate salt of the compound of Formula I, wherein Form A has an X-ray powder diffraction (XRPD) pattern as substantially shown in FIG. 1. Specifically, Form A is characterized by the following X-ray powder diffraction peaks obtained with a Cu_{Ka} radiation at 2θ (2 Theta): 9.8, 13.4, 14.2, 18.1, 18.9, 19.6, 22.6, 22.9, 25.7, 27.1, and 29.9 (±0.2°). The crystalline Form A typically has a Tₘ of about 180-190° C. by differential scanning calorimetry (DSC) analysis.

In some embodiments, treating uses a composition comprising a crystalline monohydrate form (Form B) of a monosulfate salt of the compound of Formula I, wherein Form B has an XRPD pattern as substantially shown in FIG. 2. The crystalline Form B typically has a Tₘ of about 60-70° C. by DSC analysis.

In some embodiments, treating uses a composition comprising a crystalline hemihydrate form (Form C) of a hemisulfate salt of the compound of Formula I, wherein Form C has an XRPD pattern as substantially shown in FIG. 3. The crystalline Form C typically has a Tₘ of about 90-100° C. by DSC analysis.

In some embodiments, the composition used is a tablet formulation such as a modified release tablet formulation, or a matrix pellet formulation such as a modified release matrix pellet formulation, which can be encapsulated in a capsule, as defined herein.

In some embodiments, treating uses a composition comprising a pharmaceutically acceptable salt of the compound of Formula I, wherein said salt is 90% by weight or more (e.g., 95% by weight or more, or 99% by weight or more) in crystalline Form A based on the total weight of the salt present in the composition.

The present disclosure also includes a solid pharmaceutical composition comprising a solid form of a compound of Formula I:

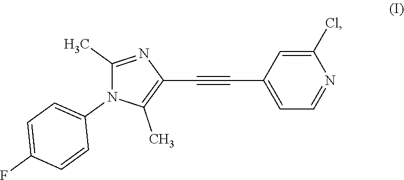 [SMILES: Cc1nc(C#Cc2ccnc(Cl)c2)c(C)n1-c1ccc(F)cc1]

wherein the solid form is a crystalline anhydrate form (Form A) of a monosulfate salt of the compound of Formula I, characterized by at least three peaks selected from the following XRPD peaks obtained with a Cu_{Ka} radiation at 2θ (2 Theta): 9.8, 13.4, 14.2, 18.1, 18.9, 19.6, 22.6, 22.9, 25.7, 27.1, and 29.9 (±0.2°); and has a median particle size (Dv50) of less than or equal to about 100 μm, wherein the solid pharmaceutical composition is the form of a matrix pellet. In some embodiments, the solid form has a particle size of less than 47 μm (e.g., about 25 μm or less, or about 10 μm or less).

In some embodiments, the pharmaceutical composition comprises the Form A monosulfate salt characterized by the following XRPD peaks obtained with a Cu_{Ka} radiation at 2θ (2 Theta): 9.8, 13.4, 14.2, 18.1, 18.9, 19.6, 22.6, 22.9, 25.7, 27.1, and 29.9 (±0.2°).

In some embodiments, the pharmaceutical composition comprises the Form A monosulfate salt characterized by an XRPD pattern as substantially shown in FIG. 1.

The present disclosure also includes a method of preparing a matrix pellet comprising a crystalline anhydrate form (Form-A) of a monosulfate salt of a compound of Formula I:

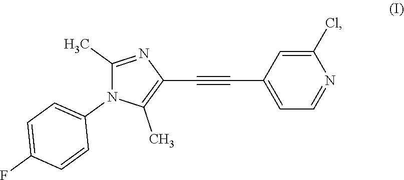 [SMILES: Cc1nc(C#Cc2ccnc(Cl)c2)c(C)n1-c1ccc(F)cc1]

wherein the method comprises: granulating the Form A monosulfate salt and one or more polymers with purified water to form a mixture; extruding, spheronizing, drying, and sieving the mixture to afford a solid material; and blending the solid material with another pharmaceutical excipient to afford a matrix pellet.

In some embodiments, the method of preparation further comprises filling the matrix pellet into a capsule to form a matrix pellet capsule. In some embodiments, the one or more polymers are selected from the group consisting of a cellulose such as microcrystalline cellulose, methacrylic acid copolymer, and hypromellose. In some embodiments, the other pharmaceutical excipient is talc.

In another aspect, provided herein are methods of treating TGN, comprising administering to a subject in need thereof a composition containing a therapeutically effective amount of an mGlu5 negative allosteric modulator (NAM) or a pharmaceutically acceptable salt thereof, wherein the mGlu5 NAM is a compound of Formula I:

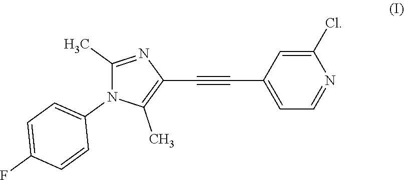 [SMILES: Cc1nc(C#Cc2ccnc(Cl)c2)c(C)n1-c1ccc(F)cc1]

The details of one or more embodiments of the disclosure are set forth in the description below. Other features, objects, and advantages of the disclosure will be apparent from the below drawings, description and from the claims.

### BRIEF DESCRIPTION OF THE DRAWINGS

FIG. 1 depicts an exemplary XRPD pattern of a crystalline anhydrate form (Form A) of a monosulfate of the compound of Formula I.

FIG. 2 depicts an exemplary XRPD pattern of a crystalline monohydrate form (Form B) of a monosulfate of the compound of Formula I.

FIG. 3 depicts an exemplary XRPD pattern of a crystalline hemihydrate form (Form C) of a hemisulfate of the compound of Formula I.

### DETAILED DESCRIPTION

As generally described herein, the present disclosure provides methods of treating trigeminal neuralgia (TGN) in a subject in need thereof. The present disclosure also describes treating TGN by using certain crystalline forms of pharmaceutically acceptable salts of the compound of Formula I. In addition, the present disclosure provides a solid form of a crystalline anhydrate form (Form A) of a monosulfate salt of the compound of Formula I, wherein the solid form has a particle size (Dv50) of less than or equal to about 100 μm (e.g., less than 47 μm, or 10 μm or less).

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. Unless defined otherwise, all abbreviations used herein have their conventional meaning within the chemical and biological arts. The chemical structures and formulae set forth herein are constructed according to the standard rules of chemical valency known in the chemical arts.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from the group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “comprise,” “comprises,” “comprising,” “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “composition” or “pharmaceutical composition” or “pharmaceutical formulation” refers to the combination of an active agent with an excipient or a carrier, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or a human, as appropriate, and/or that are approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., the compound of Formula I), which salt is compatible with pharmaceutical administration.

Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, monosulfate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, Pa. (1975).

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration, transmucosal administration (e.g., buccal, sublingual, nasal, or transdermal), or subcutaneous administration, or the implantation of a slow-release device, e.g., a mini-osmotic pump, to a subject. Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc.

By “co-administer,” it is meant that a composition described herein is administered at the same time, just prior to, or just after the administration of one or more additional therapies (e.g., anti-cancer agent, chemotherapeutic, or treatment for a neurodegenerative disease). The compound of formula I, or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, and unless otherwise specified, the terms “treat,” “treating” and “treatment” include an action that occurs while a subject is suffering from the specified disease, disorder or condition, which reduces the severity of the disease, disorder or condition, or retards or slows the progression of the disease, disorder or condition (e.g., “therapeutic treatment”). “Treat,” “treating” and “treatment”, as used herein, can include any effect, for example, lessening, reducing, modulating, ameliorating, or eliminating, that results in the improvement of the condition, disease, disorder, and the like, including one or more symptoms thereof. Treating can be curing, improving, or at least partially ameliorating the disorder.

The phrase “therapeutically effective amount,” as used herein, refers to the amount of a compound (e.g., a compound of Formula I), or a pharmaceutically acceptable salt thereof, that will elicit the biological or medical response of a tissue, system, animal or human that is being sought by the researcher, veterinarian, medical doctor or other clinician. The compound, or a pharmaceutically acceptable salt thereof, described in the present disclosure can be administered in therapeutically effective amounts to treat a disease. A therapeutically effective amount of a compound, or a pharmaceutically acceptable salt thereof, can be the quantity required to achieve a desired therapeutic and/or prophylactic effect, such as an amount which results in lessening of a symptom of a disease such as TGN.

Trigeminal neuralgia (TGN) is a long-term pain disorder that affects the trigeminal nerve. TGN, typically caused by a blood vessel compressing the trigeminal nerve, is characterized by episodes of severe facial pain along the trigeminal nerve, which is a paired cranial nerve that has three major branches: the ophthalmic nerve (V₁), the maxillary nerve (V₂), and the mandibular nerve (V₃). Although all three branches of the nerve may be affected, TGN most commonly involves the middle branch (the maxillary nerve or V₂) and lower branch (mandibular nerve or V₃) of the trigeminal nerve.

Current treatment of TGN includes microvascular decompression surgery in certain cases and use of medications such as anticonvulsants (e.g., carbamazepine) or antidepressants (e.g., amitriptyline). The existing treatment may either exhibit less favorable effectiveness or provide limited benefits due to significant side effects.

### Compound

The compound of Formula I, as depicted below, is an mGlu5 negative allosteric modulator (NAM), also known as 2-chloro-4-[1-(4-fluorophenyl)-2,5-dimethyl-1H-imidazol-4-ylethynyl]pyridine:

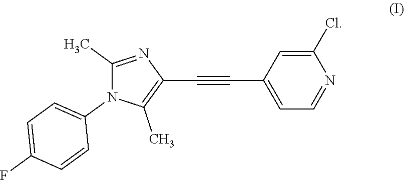 [SMILES: Cc1nc(C#Cc2ccnc(Cl)c2)c(C)n1-c1ccc(F)cc1]

A method of chemically synthesizing the compound of Formula I (including Example 1 provided herein) is described in U.S. Pat. No. 7,332,510, which is incorporated by reference in its entirety. In some embodiments, the compound of Formula I is referred to as Basimglurant.

It should be understood that the compound of Formula I as described herein includes crystalline solid forms of either the free base or pharmaceutically acceptable salts of the compound of Formula I as described herein.

In certain embodiments, the pharmaceutically acceptable salt of the compound of Formula I can be a salt of the compound of Formula I with physiologically compatible mineral acids, such as hydrochloric acid, sulfuric (or sulphuric) acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid. An exemplary pharmaceutically acceptable salt of the compound of Formula I is a monosulfate salt or a hemisulfate salt.

In certain embodiments, the pharmaceutically acceptable salt of the compound of Formula I is a monosulfate salt or a hemisulfate salt, each being in hydrate or anhydrate form (e.g., anhydrate, hemihydrate, or monohydrate).

In certain embodiments, the pharmaceutically acceptable salt of the compound of Formula I is in a crystalline form or an amorphous form.

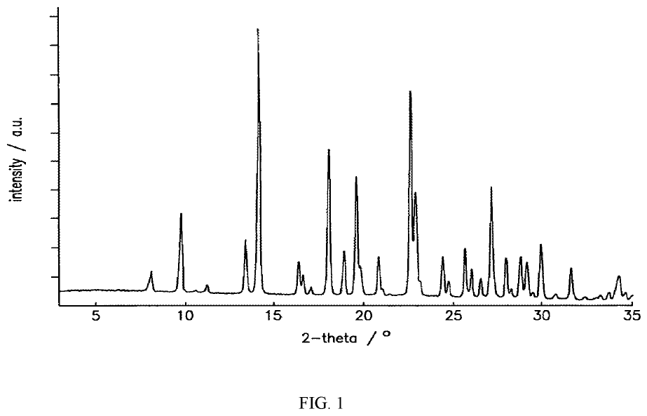

In some embodiments, the compound is in a crystalline anhydrate form (Form A) of a monosulfate salt of the compound of Formula I, wherein Form A has an X-ray powder diffraction (XRPD) pattern as substantially shown in FIG. 1. In some embodiments, Form A is characterized by at least three peaks selected from the following X-ray powder diffraction peaks obtained with a Cu_{Ka} radiation at 2θ (2 Theta): 9.8, 13.4, 14.2, 18.1, 18.9, 19.6, 22.6, 22.9, 25.7, 27.1, and 29.9 (±0.2°). The crystalline Form A typically has a Tₘ of about 180-190° C. by DSC analysis. In some embodiments, Form A is characterized by an infrared spectrum having sharp bands at 3068, 2730, 2618, 2236, 2213, 1628, 1587, 1569, 1518, 1384, 1374, 1295, 1236, 1168, 1157, 1116, 1064, 1019, 902, 855, 786, and 674 cm⁻¹ (±3 cm⁻¹).

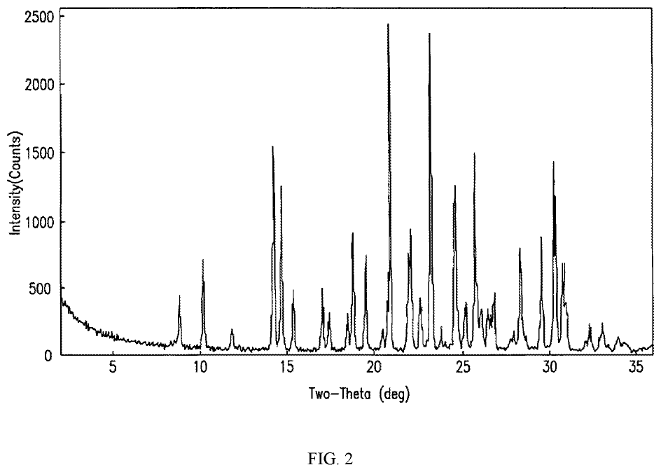

In some embodiments, the compound is in a crystalline monohydrate form (Form B) of a monosulfate salt of the compound of Formula I, wherein Form B has an XRPD pattern as substantially shown in FIG. 2. The crystalline Form B typically has a Tₘ of about 60-70° C. by DSC analysis.

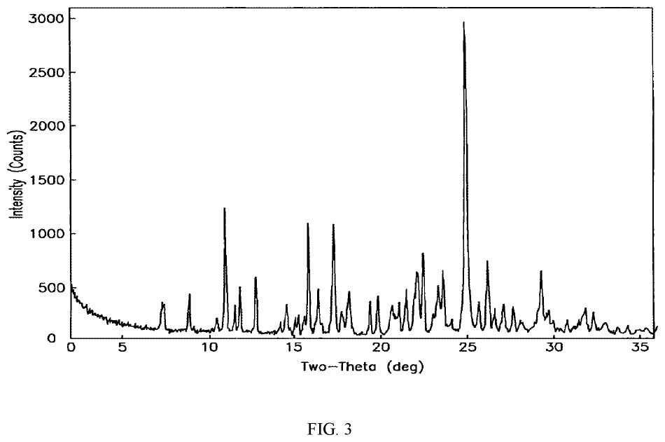

In some embodiments, the compound is in a crystalline hemihydrate form (Form C) of a hemisulfate salt of the compound of Formula I, wherein Form C has an XRPD pattern as substantially shown in FIG. 3. The crystalline Form C typically has a Tₘ of about 90-100° C. by DSC analysis.

### Pharmaceutical Compositions

In one aspect, the present disclosure relates to a composition such as a pharmaceutical composition comprising the compound of Formula I, or a pharmaceutically acceptable salt thereof, and a pharmaceutically acceptable excipient, for the treatment of TGN in a subject in need thereof. In various embodiments, the composition is a solid pharmaceutical composition.

In various embodiments, the amount of the compound of Formula I, or a pharmaceutically acceptable salt thereof, in the pharmaceutical compositions described herein can be from about 0.01 mg to about 30 mg, about 0.05 mg to about 20 mg, about 0.1 mg to about 10 mg, about 0.5 mg to about 10 mg, about 1 mg to about 10 mg, about 2 mg to about 10 mg, about 3 mg to about 10 mg, about 4 mg to about 10 mg, about 5 mg to about 10 mg, about 6 mg to about 10 mg, about 7 mg to about 10 mg, about 8 mg to about 10 mg, about 9 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4.5 mg, about 1 mg to about 4 mg, about 1 mg to about 3.5 mg, about 1 mg to about 3 mg, about 1 mg to about 2.5 mg, about 1 mg to about 2 mg, about 1 mg to about 1.5 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4.5 mg, about 1.5 mg to about 4 mg, about 1.5 mg to about 3.5 mg, about 1.5 mg to about 3 mg, about 1.5 mg to about 2.5 mg, about 1.5 mg to about 2 mg, about 2 mg to about 9 mg, about 2 mg to about 8 mg, about 2 mg to about 7 mg, about 2 mg to about 6 mg, about 2 mg to about 5 mg, about 2 mg to about 4.5 mg, about 2 mg to about 4 mg, about 2 mg to about 3.5 mg, about 2 mg to about 3 mg, about 2 mg to about 2.5 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4.5 mg, about 2.5 mg to about 4 mg, about 2.5 mg to about 3.5 mg, about 2.5 mg to about 3 mg, about 3 mg to about 9 mg, about 3 mg to about 8 mg, about 3 mg to about 7 mg, about 3 mg to about 6 mg, about 3 mg to about 5 mg, about 3 mg to about 4.5 mg, about 3 mg to about 4 mg, about 3 mg to about 3.5 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 4.5 mg, about 3.5 mg to about 4 mg, about 4 mg to about 9 mg, about 4 mg to about 8 mg, about 4 mg to about 7 mg, about 4 mg to about 6 mg, about 4 mg to about 5 mg, about 4 mg to about 4.5 mg, about 4.5 mg to about 9 mg, about 4.5 mg to about 8 mg, about 4.5 mg to about 7 mg, about 4.5 mg to about 6 mg, about 4.5 mg to about 5 mg, about 5 mg to about 9 mg, about 5 mg to about 8 mg, about 5 mg to about 7 mg, about 5 mg to about 6 mg, about 6 mg to about 9 mg, about 6 mg to about 8 mg, about 6 mg to about 7 mg, about 7 mg to about 9 mg, about 7 mg to about 8 mg, or about 8 mg to about 9 mg.

In certain embodiments, the amount of the compound of Formula I, or a pharmaceutically acceptable salt thereof, in the pharmaceutical compositions described herein can be from about 0.1 mg to about 1.5 mg.

In certain embodiments, the amount of the compound of Formula I, or a pharmaceutically acceptable salt thereof, in the pharmaceutical compositions described herein can be from about 0.1 mg to about 4.0 mg, from about 0.1 mg to about 3.5 mg, from about 0.1 mg to about 3.0 mg, from about 1.5 mg to about 3.5 mg, or from about 1.0 mg to about 3.0 mg once daily. In certain embodiments, the amount of the compound of Formula I, or a pharmaceutically acceptable salt thereof, in the pharmaceutical compositions described herein can be an amount of about 4.0 mg, about 3.5 mg, about 3.0 mg, about 2.5 mg, about 2.0 mg, about 1.5 mg, or about 1.0 mg once daily.

In certain embodiments, the amount of the compound of Formula I, or a pharmaceutically acceptable salt thereof, in the pharmaceutical compositions described herein is about 4.0 mg. In certain embodiments, the amount of the compound of Formula I, or a pharmaceutically acceptable salt thereof, in the pharmaceutical compositions described herein is about 3.5 mg. In certain embodiments, the amount of the compound of Formula I, or a pharmaceutically acceptable salt thereof, in the pharmaceutical compositions described herein is about 3.0 mg. In certain embodiments, the amount of the compound of Formula I, or a pharmaceutically acceptable salt thereof, in the pharmaceutical compositions described herein is about 2.5 mg. In certain embodiments, the amount of the compound of Formula I, or a pharmaceutically acceptable salt thereof, in the pharmaceutical compositions described herein is about 2.0 mg. In certain embodiments, the amount of the compound of Formula I, or a pharmaceutically acceptable salt thereof, in the pharmaceutical compositions described herein is about 1.5 mg. In certain embodiments, the amount of the compound of Formula I, or a pharmaceutically acceptable salt thereof, in the pharmaceutical compositions described herein is about 1.0 mg.

In various embodiments, the amount of the compound of Formula I, or a pharmaceutically acceptable salt thereof, in the pharmaceutical compositions described herein can be about 0.1 mg, about 0.2 mg, about 0.3 mg, about 0.4 mg, about 0.5 mg, about 1.0 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg.

In certain embodiments, the amount of the compound of Formula I, or a pharmaceutically acceptable salt thereof, in the pharmaceutical compositions described herein can be about 0.1 mg to about 0.2 mg (e.g., about 0.13 mg).

In certain embodiments, the amount of the compound of Formula I, or a pharmaceutically acceptable salt thereof, in the pharmaceutical compositions described herein can be about 0.2 mg to about 0.3 mg (e.g., about 0.26 mg).

In certain embodiments, the amount of the compound of Formula I, or a pharmaceutically acceptable salt thereof, in the pharmaceutical compositions described herein can be about 0.6 mg to about 0.7 mg (e.g., about 0.65 mg).

In certain embodiments, the amount of the compound of Formula I, or a pharmaceutically acceptable salt thereof, in the pharmaceutical compositions described herein can be about 1.2 mg to about 1.4 mg (e.g., about 1.3 mg).

In some embodiments, the compound of Formula I, or a pharmaceutically acceptable salt thereof, is present in the composition in an amount from about 0.01% to about 20% by weight, about 0.05% to about 15% by weight, about 0.1% to about 10% by weight, about 0.1% to about 5% by weight, about 0.1% to about 1% by weight, or about 0.1% to about 0.5% by weight, based on the total weight of the composition.

In some embodiments, the compound of Formula I, or a pharmaceutically acceptable salt thereof, is present in the composition in an amount from about 0.05% to about 15% by weight.

In some embodiments, the compound of Formula I, or a pharmaceutically acceptable salt thereof, is present in the composition in an amount from about 0.1% to about 0.5% by weight.

In various embodiments, the pharmaceutical compositions described herein comprise a therapeutically effective amount of a pharmaceutically acceptable salt of the compound of Formula I. In some embodiments, the pharmaceutically acceptable salt of the compound of Formula I can be a salt of the compound of Formula I with physiologically compatible mineral acids, such as hydrochloric acid, sulfuric acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid.

In certain embodiments, the pharmaceutically acceptable salt of the compound of Formula I is a monosulfate salt or a hemisulfate salt, each being in hydrate or anhydrate form (e.g., anhydrate, hemihydrate, or monohydrate).

In certain embodiments, the pharmaceutically acceptable salt of the compound of Formula I is in a crystalline form or an amorphous form.

In certain embodiments, the pharmaceutical compositions described herein comprise a crystalline anhydrate form (Form A) of a monosulfate salt of the compound of Formula I, wherein Form A has an X-ray powder diffraction (XRPD) pattern as substantially shown in FIG. 1. The crystalline Form A typically has a Tₘ of about 180-190° C. by DSC analysis (e.g., 180±1° C., 182±1° C., 184±1° C., 186±1° C., 188±1° C., or 190±1° C.).

In some embodiments, the pharmaceutical compositions described herein comprise a crystalline monohydrate form (Form B) of a monosulfate salt of the compound of Formula I, wherein Form B has an XRPD pattern as substantially shown in FIG. 2. The crystalline Form B typically has a Tₘ of about 60-70° C. by DSC analysis (e.g., 60±1° C., 62±1° C., 64±1° C., 66±1° C., 68±1° C., or 70±1° C.).

In some embodiments, the pharmaceutical compositions described herein comprise a crystalline hemihydrate form (Form C) of a hemisulfate salt of the compound of Formula I, wherein Form C has an XRPD pattern as substantially shown in FIG. 3. The crystalline Form C typically has a Tₘ of about 90-100° C. by DSC analysis (e.g., 90±1° C., 92±1° C., 94±1° C., 96±1° C., 98±1° C., or 100±1° C.).

In certain embodiments, the pharmaceutical compositions described herein comprise an amorphous form of a monosulfate salt of the compound of Formula I, wherein said amorphous form is characterized by an infrared spectrum having bands at 2730, 2592, 2219, 1633, 1586, 1570, 1513, 1375, 1343, 1293, 1226, 1157, 1130, 1084, 1040, 986, 903, 848, 788, 712 and 670 cm⁻¹ (+3 cm⁻¹).

In some embodiments, the pharmaceutical composition is a tablet formulation such as a modified release tablet formulation, or a matrix pellet formulation such as a modified release matrix pellet formulation, which can be encapsulated in a capsule. A “modified release formulation,” or a “modified-release dosage,” as used herein, refers to a mechanism that (in contrast to immediate-release dosage) delivers a drug with a delay after its administration (delayed-release dosage), or for a prolonged period of time (extended-release dosage). See, Perrie et al., Pharmaceutics: Drug Delivery and Targeting (2^{nd}), 2012, 7-13.

In certain embodiments, the pharmaceutical composition is a modified release matrix pellet formulation encapsulated in a capsule, wherein the compound of Formula I, or a pharmaceutically acceptable salt thereof, is present in an amount from about 0.05 mg to about 20 mg (e.g., about 0.1 mg to about 0.2 mg, about 0.2 mg to about 0.3 mg, about 0.6 mg to about 0.7 mg, or about 1.2 mg to about 1.4 mg).

In certain embodiments, the pharmaceutical composition is a modified release pellet formulation encapsulated in a capsule, wherein the compound of Formula I, or a pharmaceutically acceptable salt thereof, is present in the composition in an amount from about 0.01% to about 20% by weight (e.g., about 0.05% to about 15% by weight, about 0.1% to about 1% by weight, or about 0.1% to about 0.5% by weight), based on the total weight of the composition.

In certain embodiments, the pharmaceutical composition described herein comprises a crystalline anhydrate form (Form A) of a monosulfate salt of the compound of Formula I, wherein Form A has an XRPD pattern as substantially shown in FIG. 1; and the Form A monosulfate salt is present in the composition in an amount from about 0.05 mg to about 20 mg (e.g., about 0.1 mg to about 0.2 mg, about 0.2 mg to about 0.3 mg, about 0.6 mg to about 0.7 mg, or about 1.2 mg to about 1.4 mg).

In certain embodiments, the pharmaceutical composition described herein comprises a crystalline anhydrate form (Form A) of a monosulfate salt of the compound of Formula I, wherein Form A has an XRPD pattern as substantially shown in FIG. 1; and the Form A monosulfate salt is present in the composition in an amount from about 0.01% to about 20% by weight (e.g., about 0.05% to about 15% by weight, about 0.1% to about 1% by weight, or about 0.1% to about 0.5% by weight), based on the total weight of the composition.

As described herein, the present disclosure includes a solid pharmaceutical composition comprising a solid form of a compound of Formula I, namely, a crystalline anhydrate form (Form A) of a monosulfate salt of the compound of Formula I; where the composition is in the form of a matrix pellet and the solid form has a mean particle size (Dv50) of less than or equal to about 100 μm. The Dv50 can be determined by a LA-950 laser diffraction method. See, e.g., https://static.horiba.com/fileadmin/Horiba/Products/Scientific/Particle_Characterization/Downloads/Technica_Notes/TN159_LA-950_Laser_Diffraction_Technique.pdf.

In various embodiments, the pharmaceutical composition comprises a matrix pellet of the solid form having a particle size of less than 47 μm, less than 45 μm, less than 40 μm, less than 35 μm, less than 30 μm, less than 25 μm, less than 20 μm, less than 15 μm, less than 10 μm, or less than 5 μm. In certain embodiments, the solid form has a particle size of about 10 μm or less (e.g., about 10 μm, about 9 μm, about 8 μm, about 7 μm, about 6 μm, about 5 μm, about 4 μm, about 3 μm, about 2 μm, or about 1 μm).

In certain embodiments, the solid form has a particle size of less than 47 μm and the Form A monosulfate salt is present in the composition in an amount of 1% by weight or less, based on the total weight of the composition.

In certain embodiments, the solid form has a particle size of about 10 μm or less (e.g., about 3.3 μm), and the Form A monosulfate salt is present in the composition in an amount of 0.5% by weight or less (e.g., 0.1% by weight), based on the total weight of the composition.

In certain embodiments, the pharmaceutical composition comprises a pharmaceutical excipient comprising a polymer, a binder, a disintegrant, a lubricant, or a glidant.

In certain embodiments, the polymer is a matrix forming polymer (e.g., microcrystalline cellulose), a pH-responding polymer (e.g., methacrylic acid copolymer), or a binder (e.g., hypromellose). In certain embodiments, the polymer is one or more polymers selected from the group consisting of a cellulose such as microcrystalline cellulose, methacrylic acid copolymer, and hypromellose. In certain embodiments, the glidant is talc.

The pharmaceutical compositions provided herein can be administered by a variety of routes including, but not limited to, oral administration, administration as a suppository, topical contact, parenteral administration (e.g., intravenous, intramuscular, intra-arterial, intradermal, subcutaneous, intraperitoneal, intraventricular, and intracranial), intralesional administration, intrathecal administration, intranasal administration, transmucosal administration (e.g., buccal, sublingual, nasal, or transdermal), or the implantation of a slow-release device, e.g., a mini-osmotic pump, to a subject. In certain embodiments, the pharmaceutical compositions disclosed herein are administered orally.

The pharmaceutical compositions provided herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions provided herein may be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. In various embodiments, the pharmaceutical dosage forms described herein can be administered as a unit dose. Typical unit dosage forms include prefilled, premeasured ampules or syringes of the liquid compositions or pills, tablets, capsules or the like in the case of solid compositions.

In certain embodiments, the pharmaceutical compositions provided herein are administered to the patient as a solid dosage form. In certain embodiments, the solid dosage form is a capsule (e.g., a modified release pellet formulation encapsulated in a capsule). In certain embodiments, the solid dosage form is a tablet (e.g., a modified release tablet formulation).

In certain embodiments, the pharmaceutical compositions provided herein comprise the compound of Formula I as the sole active agent, or in combination with other active agents.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

In one aspect, provided herein are methods of treating trigeminal neuralgia (TGN) in a subject (e.g., a human) in need thereof.

In various embodiments, provided herein are methods for treating TGN in a subject in need thereof, comprising administering to a subject in need thereof a composition comprising a therapeutically effective amount of a compound or a pharmaceutically acceptable salt thereof, wherein the compound is of Formula I:

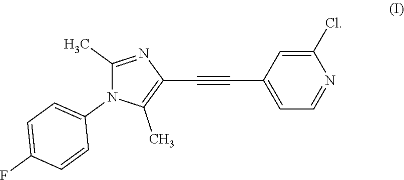 [SMILES: Cc1nc(C#Cc2ccnc(Cl)c2)c(C)n1-c1ccc(F)cc1]

In certain embodiments, provided herein are methods of treating TGN in a subject in need thereof, comprising administering to a subject in need thereof a composition comprising a therapeutically effective amount of a compound or a pharmaceutically acceptable salt thereof, wherein the compound is of Formula I:

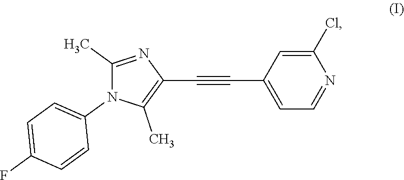 [SMILES: Cc1nc(C#Cc2ccnc(Cl)c2)c(C)n1-c1ccc(F)cc1]

wherein administering comprises administering the compound of Formula I, or a pharmaceutically acceptable salt thereof, in an amount of about 1.5 mg to about 3.5 mg once daily. In some embodiments, the subject has a weight of at least about 71 kg. In some embodiments, the subject has a weight less than about 71 kg (e.g., 30, 35, 40, 45, 50, 55, 60, 65, or 70 kg). In some embodiments, the subject has a weight of at least about 71 kg. In some embodiments, the subject has a weight less than about 71 kg (e.g., 30, 35, 40, 45, 50, 55, 60, 65, or 70 kg). In some embodiments, the subject has a weight from about 71 kg to about 150 kg (e.g., 70, 71, 75, 80, 85, 90, 95, 100, 105, 110, 115, 120, 125, 130, 135, 140, 145, or 150 kg).

In various embodiments, administering a therapeutically effective amount of the compound of Formula I, or a pharmaceutically acceptable salt thereof, comprises administering to a subject from about 0.05 mg to about 20 mg (e.g., about 0.1 mg, about 0.2 mg, about 0.3 mg, about 0.4 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg) of the compound of Formula I, or a pharmaceutically acceptable salt thereof.

In various embodiments, administering a therapeutically effective amount of the compound of Formula I, or a pharmaceutically acceptable salt thereof, comprises administering to a subject from about 0.1 mg to about 4.0 mg, from about 0.1 mg to about 3.5 mg, from about 0.1 mg to about 3.0 mg, from about 1.5 mg to about 3.5 mg, or from about 1.0 mg to about 4.0 mg. In certain embodiments, administering a therapeutically effective amount of the compound of Formula I, or a pharmaceutically acceptable salt thereof, comprises administering to a subject from about 1.5 mg to about 3.5 mg.

In certain embodiments, administering a therapeutically effective amount of the compound of Formula I, or a pharmaceutically acceptable salt thereof, comprises administering to a subject about 1.0 mg once daily. In certain embodiments, administering a therapeutically effective amount of the compound of Formula I, or a pharmaceutically acceptable salt thereof, comprises administering to a subject about 1.5 mg once daily. In certain embodiments, administering a therapeutically effective amount of the compound of Formula I, or a pharmaceutically acceptable salt thereof, comprises administering to a subject about 2.0 mg once daily. In certain embodiments, administering a therapeutically effective amount of the compound of Formula I, or a pharmaceutically acceptable salt thereof, comprises administering to a subject about 2.5 mg once daily. In certain embodiments, administering a therapeutically effective amount of the compound of Formula I, or a pharmaceutically acceptable salt thereof, comprises administering to a subject about 3.0 mg once daily. In certain embodiments, administering a therapeutically effective amount of the compound of Formula I, or a pharmaceutically acceptable salt thereof, comprises administering to a subject about 3.5 mg once daily. In certain embodiments, administering a therapeutically effective amount of the compound of Formula I, or a pharmaceutically acceptable salt thereof, comprises administering to a subject about 4.0 mg once daily. In certain embodiments, administering a therapeutically effective amount of the compound of Formula I, or a pharmaceutically acceptable salt thereof, comprises administering from about 0.1 mg to about 1.5 mg of the compound of Formula I, or a pharmaceutically acceptable salt thereof, to a subject in need thereof.

In certain embodiments, provided herein is a method of administering the free base form of the compound of Formula I for the treatment of TGN in a subject in need thereof.

In certain embodiments, provided herein is a method of administering a pharmaceutically acceptable salt of the compound of Formula I for the treatment of TGN in a subject in need thereof.

In certain embodiments, treating comprises administering the compound of Formula I, or a pharmaceutically acceptable salt thereof, once, twice, three, four, or five times daily. In certain embodiments, treating comprises administering the compound of Formula I, or a pharmaceutically acceptable salt thereof, once daily.

In certain embodiments, treating comprises administering the compound of Formula I, or a pharmaceutically acceptable salt thereof, by a variety of routes including, but not limited to, oral administration, administration as a suppository, topical contact, parenteral administration (e.g., intravenous, intramuscular, intra-arterial, intradermal, subcutaneous, intraperitoneal, intraventricular, and intracranial), intralesional administration, intrathecal administration, intranasal administration, transmucosal administration (e.g., buccal, sublingual, nasal, or transdermal), or the implantation of a slow-release device, e.g., a mini-osmotic pump, to a subject.

In certain embodiments, treating comprises administering the compound of Formula I, or a pharmaceutically acceptable salt thereof, by oral administration.

In certain embodiments, treating comprises administering the compound of Formula I, or a pharmaceutically acceptable salt thereof, as a unit dose.

In certain embodiments, treating comprises administering the compound of Formula I as the free base form.

In certain embodiments, treating comprises administering the compound of Formula I in the form of a pharmaceutically acceptable salt thereof. In some embodiments, the pharmaceutically acceptable salt of the compound of Formula I can be a salt of the compound of Formula I with physiologically compatible mineral acids, such as hydrochloric acid, sulfuric (or sulphuric) acid, sulphurous acid or phosphoric acid; or with organic acids, such as methanesulphonic acid, p-toluenesulphonic acid, acetic acid, lactic acid, trifluoroacetic acid, citric acid, furmaric acid, maleic acid, tartaric acid, succinic acid or salicylic acid.

In certain embodiments, as described above, treating comprises administering the compound of Formula I in a crystalline form (e.g., Form A, Form B, or Form C) in a sulfate salt form (e.g., a monosulfate salt or a hemisulfate salt).

In various embodiments, provided herein are methods of treating TGN, comprising administering to a subject in need thereof a composition comprising a therapeutically effective amount of an mGlu5 negative allosteric modulator (NAM), or a pharmaceutically acceptable salt thereof, wherein the mGlu5 NAM is a compound of Formula I:

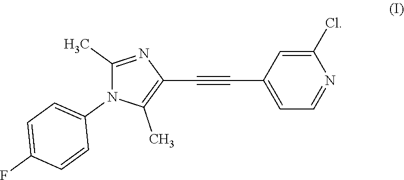 [SMILES: Cc1nc(C#Cc2ccnc(Cl)c2)c(C)n1-c1ccc(F)cc1]

In certain embodiments, treating comprises administering the compound of Formula I as a monotherapy.

In certain embodiments, the methods provided herein further comprise administering a therapeutically effective amount of another therapeutic agent to the subject.

As described herein, the present invention relates to use of the compound of Formula I, or a pharmaceutically acceptable salt thereof, for treating trigeminal neuralgia (TGN). In some embodiments, the TGN is typical TGN, which results in episodes of severe, sudden, shock-like pain in one side of the face that lasts for seconds to a few minutes. In some embodiments, the TGN is atypical TGN, which results in a constant burning pain that is less severe. In certain embodiments, the TGN is classified by the International Classification of Headache (ICHD-3). In certain embodiments, the TGN is a classical TGN. In some embodiments, the TGN is a secondary TGN. In certain embodiments, the TGN is a idiopathic TGN.

In certain embodiments, the therapeutic effect of the treatment is determined by:
a) reduction of the activity of high-voltage activated calcium channels;
b) reduction of the activity of voltage-gated sodium channels;
c) suppression of the propagation of an ion channel action potential; or
d) suppression of the rapid firing of neurons.

In some embodiments, the efficacy of the compound is determined by Brief Pain Inventory-Facial (BPI-F) scale. In some embodiments, the efficacy of the compound is determined by Global Impression of change. In some embodiments, the efficacy of the compound is determined by Sheehan Disability Scale (SDS). In some embodiments, the efficacy of the compound is determined by patient diary cards. In some embodiments, the efficacy of the compound is determined by number and severity of attacks (paroxysms) or number of pain free days. In some embodiments, the efficacy of the compound is determined by rating of the Medication Satisfaction Questionnaire (MSQ). In some embodiments, the efficacy of the compound is determined by Patient Global Impression of Change (PGI-C). In some embodiments, the efficacy of the compound is determined by patient-rated Global Impression-severity.

Without further elaboration, it is believed that one skilled in the art can, based on the above description, utilize the present invention to its fullest extent. The following specific examples are therefore to be construed as merely illustrative, and not limitative of the remainder of the disclosure in any way whatsoever. All publications cited herein are incorporated by reference in their entirety.

### EXAMPLES

In order that the disclosure described herein may be more fully understood, the following examples are set forth. The examples described in this application are offered to illustrate the compound, pharmaceutical compositions, and methods described herein and are not to be construed in any way as limiting their scope.

### Example 1: Synthesis of 2-chloro-4-[1-(4-fluorophenyl)-2,5-dimethyl-1H-imidazol-4-ylethynyl]pyridine (Compound of Formula I, supra) [See U.S. Pat. No. 7,332,510]

2-Chloro-4-[1-(4-fluorophenyl)-2-methyl-1H-imidazol-4-ylethynyl]-pyridine (200 mg, 0.6 mmol) was dissolved in 10 mL tetrahydrofuran (TIF) and cooled to −75° C. Lithiumdiisopropylamide (0.45 mL, 0.91 mmol) was added and the mixture stirred for 15 min at −75° C. Iodomethane (0.05 mL, 0.85 mmol) was added and stirring was continued at −75° C. for 2 hrs. The reaction mixture was quenched with saturated NaHCO₃ solution and extracted with water and ethyl acetate. The combined organic extracts were dried with sodium sulfate, filtered and evaporated. The crude product was purified by flash chromatography on silica gel (heptane/ethyl acetate 90:10 to 20:80 gradient) and by recrystallization from ethyl acetate. The title compound was obtained as a white solid. MS: m/z=326.5 (M+H+).

### Example 2: Preparation of Polymorphs of Salts of Compound of Formula I [See U.S. Pat. No. 8,063,076]

Form A monosulfate salt: 61.0 g of 2-chloro-4-[1-(4-fluoro-phenyl)-2,5-dimethyl-1H-imidazol-4-ylethynyl]-pyridine was dissolved in 610 mL of 2-propanol. The solution was filtered and the filter rinsed with 31 mL of 2-propanol. To the combined solutions a mixture of 30 mL of water and 18.91 g of sulfuric acid (97%) was added drop-wise. The solution was cooled to 0-5° C. Seeding was performed at 58° C. as needed. The solid residues were filtered, washed with 2-propanol (0-5° C.) and dried at 50° C. and less than 1 mbar for 18 hrs to provide the monosulfate salt of the compound of Formula I in a yield of 69.1 g (87.1%). Form A seeding crystals can be prepared upon cooling crystallization of a hot solution of 250 mg of the monosulfate salt in 10 mL of 2-propanol. After cooling to 0° C., the solid residues can be filtered and dried at 50° C. under vacuum to afford Form A monosulfate salt, which was confirmed by the XRPD pattern as substantially shown in FIG. 1.

Form B monosulfate salt: 300 mg of Form A monosulfate salt of the compound of Formula I was dissolved in 3 mL 2-propanol and 1 mL water at 60° C. to produce a clear solution. The clear solution was seeded with Form B monosulfate salt and sealed at room temperature (e.g., about 25° C.). Single crystals were formed after 3 days. Seeding crystals can be prepared by formation of a saturated slurry of Form A monosulfate salt of the compound of Formula I in 2-propanol and water (3:1 v/v) at room temperature. The slurry was stirred at room temperature for approximately 3 weeks. The solids were filtered via a glass 35 filter to afford crystalline Form B monosulfate salt, which was confirmed by the XRPD pattern as substantially shown in FIG. 2.

Form C hemisulfate salt: 41 g of Form A monosulfate of the compound of Formula I was mixed with 128 g of water. The slurry was stirred at room temperature for 2-16 hrs. After all the Form A monosulfate salt had been converted to the hemisulfate salt, the resulting crystals were collected by filtration and rinsed with water. The wet cake thus obtained was dried at 40° C. in a vacuum oven for 48 hrs to afford Form C hemisulfate salt in a yield of 93%. Form C hemisulfate salt was confirmed by the XRPD pattern as substantially shown in FIG. 3.

Amorphous monosulfate salt: 0.53 g of a monosulfate of the compound of Formula I was dissolved in 10 mL of methanol at approximately 65° C. After complete evaporation of the solvent under vacuum, the solid (foam) was further dried at about 50° C. under 5-20 mbar for 18 hrs. Analysis (XRPD and DSC) revealed amorphous form of the compound of Formula I was obtained. The amorphous monosulfate salt was characterized by an infrared spectrum having bands at 2730, 2592, 2219, 1633, 1586, 1570, 1513, 1375, 1343, 1293, 1226, 1157, 1130, 1084, 1040, 986, 903, 848, 788, 712 and 670 cm⁻¹ (±3 cm⁻¹). The glass transition temperature (T_{g}) of the amorphous form determined by DSC was largely dependent on the solvent content and was observed for the wet sample (closed pan) at about 42° C. and for the in-situ dried sample (pan with perforation lid) at about 77° C.

### Example 3: Modified Release Matrix Pellet Capsules of Form A Monosulfate Salt

Two different matrix pellet compositions were prepared according to the formulations shown in Table 1 below. The matrix pellets thus obtained were filled into capsules to afford matrix pellet capsules. The process included: high shear wet granulating Form A monosulfate salt, microcrystalline cellulose, methacrylic acid copolymer, and hypromellose, with purified water to form a mixture; followed by extruding, spheronizing, fluid bed drying, and sieving the mixture to provide a solid material; and subsequently blending the solid material with an external pharmaceutical excipient, talc, to afford matrix pellets; and then filling the matrix pellets into capsules to provide matrix pellet capsules. More specifically, each of the granulating, extruding, spheronizing, drying, sieving, and blending steps was carried out as follows.
1. Weighed the Form A monosulfate and about 15% of the required amount of microcrystalline cellulose and place them into a suitable container. Mixed the contents using a turbula mixer or an equivalent blender for 30 minutes at 40±10 rpm.
2. Weighed all the other excipients: methacrylic acid copolymer, hypromellose, and the remaining microcrystalline cellulose.
3. Transferred all the materials from Step 2 into a high shear granulator followed by the mixture from Step 1. Mix all the components for two minutes using the impeller and chopper at the following speeds: Impeller: 300±100 rpm and Chopper: 1500±500 rpm.
4. Granulated the powder mixture from Step 3 by spraying purified water (approximately 83% of the batch size) onto the powder mixture in the high shear granulator while continually mixing the contents using the impeller at 300±100 rpm and Chopper at 1500±500 rpm for 20 minutes. Recorded the power consumption at the granulation end point.
5. Fed the wet granules at a uniform rate and extruded the wet granules through the extruder using screen #1.0 mm and speed setting of about 40±5 rpm.
6. Transferred about 700 g of the extruded material from Step 5 into a spheronizer using #1 graded plate. Spheronized the contents for 5±1 minutes at a speed of about 0.6 (approximately 1000 rpm).
7. Collected the spheronized material from Step 6 and dried in a fluid bed dryer with inlet temperature of 60±10° C., until the water content of the pellets was less than 0.8% measured using a Halogen Moisture Analyzer or an equivalent set at 90° C.
8. Screened the dried pellets from Step 7 through size #10 and #40 screens and collected the pellet fraction between #10 and #40 screen.
9. Used the weight of the pellets from Step 8 to weigh the adjusted amount of talc.
10. Placed the pellets from Step 9 in a bin blender or an equivalent, added talc and mixed for 5 minutes at 20±5 rpm.
11. Filled the pellets from Step 10 into hard gelatin capsules.
12. Stored the filled capsules from Step 11 in double polyethylene-lined bags with two silica gel bags between the polyethylene bags in a closed fiber drum at a temperature not above 25° C.

TABLE 1
Matrix Pellet Formulations

| | | |
|---|---|---|
| Ingredient | Quantity (mg/capsule) | Function |

| | | | |
|---|---|---|---|
| Form A | 0.13* | 1.30^{#} | Active |
| monosulfate | | | ingredient |
| Microcrystalline | 64.62 | 128.20 | Matrix forming |
| cellulose | | | polymer |
| Methacrylic acid | 30.00 | 60.00 | pH-responding |
| copolymer | | | polymer |
| Hypromellose | 5.00 | 10.00 | Binder |
| Talc | 0.25 | 0.50 | Glidant |
| Total | 100.00 | 200.00 | |

*Represents 0.5 mg dosage and
^{#}represents 1.0 mg dosage described in the paragraph below.

The content uniformity of the matrix pellets was found to be dependent on the median particle size (Dv50) and amount of the Form A monosulfate salt (“API”). Specifically, three API variants achieved by size reduction with jet mill and pin mill to a respective median particle size (Dv50) of 3.3 μm (et-milled), 10 μm (pin-milled), and 47 μm (pin-milled) were manufactured at two different dosages (API. 0.1 mg and 1.0 mg). At the dosage of 1.0 mg of API, matrix pellets prepared using API with Dv50 of 3.3 μm, 10 μm, and 47 μm exhibited USP uniformity of dosage units (UDU) acceptance value (AV) of 2.2, 6.3, 3.4, respectively, all meeting the UDU acceptance criteria (AV<15). At the dosage of 0.1 mg of API, matrix pellets prepared using API with Dv50 of 47 μm failed to meet the UDU acceptance criteria with an AV of 20.9; unexpectedly, matrix pellets prepared using API with Dv50 of 3.3 μm and 10 μm met the UDU acceptance criteria with an AV of 6.0 and 10.3, respectively. API particle size (Dv50) of 10 μm or less (e.g., between 3.3-10 μm) was shown to provide matrix pellets drug product with acceptable manufacturing process and drug product performance (e.g., content uniformity, pellet size distribution, and dissolution), thus more suitable for pharmaceutical drug development.

### Example 4: A Study of the Compound of Formula I for Treatment of Subjects with Trigeminal Neuralgia (TGN)

A Phase II/111, multicenter, 8-week run-in phase followed by a 12-week, prospective, parallel-group, double-blind, randomized withdrawal, placebo-controlled study, with a 52 week open label extension, was carried out, as shown in Table 2 below, to evaluate the efficacy and safety of daily 1.5 to 3.5 mg the compound of Formula I (also referred to herein as “Basimglurant”) in patients with pain associated with trigeminal neuralgia with suboptimal response to their current anti-pain therapy. The study duration was up to 24 weeks and consists of 3 periods followed by a 52 week open label extension. 200 patients were enrolled in study Period 1 to randomize 70 patients in Period 2.

TABLE 2
Objectives and endpoints

| | |
|---|---|
| Objectives | Endpoints |

PERIOD 1. RUN-IN
Primary

| | |
|---|---|
| To evaluate the safety of basimglurant daily | Incidence and severity of adverse events, |
| dosing 1.5-3.5 mg. | laboratory, vital signs and cardiovascular |
| | safety will also be evaluated. |

Exploratory

| | |
|---|---|
| To evaluate the efficacy of 8-week once daily | Mean change from Period 1 baseline (BL1) |
| treatment with basimglurant on pain | to Week 8 in the total patient-rated Brief Pain |
| associated withtrigeminal neuralgia on the | Inventory-Facial (BPI-F) scale. |
| following disease aspects: | Measure Global Impression of change from |
| Impact on Facial Pain | Period 1 baseline (BL1) to Week 8. |
| Patient perceived change of the pain | Number and severity of attacks (paroxysms) |
| Quantitative and qualitative pain | as well as duration and severity of continuous |
| assessments | pain compared with BL1. |
| Pain freedom | Number of pain free days. |
| Patient medication satisfaction | Patient reported rating of the Medication |
| | Satisfaction Questionnaire (MSQ). |

PERIOD 2. DOUBLE BLIND
Primary

| | |
|---|---|
| To assess the maintenance of effect on pain | Time to Loss of Efficacy or pain recurrence |
| of double-blind 12-week once daily dosing | defined as the confirmed increase in the |
| of basimglurant 1.5-3.5 mg compared with | number of weekly paroxysms or re- |
| placebo in patients with TGN. | emergence of continuous pain and/or the need |
| | for rescue medication |

Secondary

| | |
|---|---|
| To evaluate the effect of double-blind | Mean change in the total patient-rated Brief |
| treatment of once daily dose of basimglurant | Pain Inventory-Facial (BPI-F) scale compared |
| versus placebo on the following disease | with Period 2 baseline (BL2) |
| aspects: | Frequency and severity of attacks |
| Impact on facial pain | (paroxysms) as well as severity and duration |
| Pain frequency and severity | of continuous pain captured in patient diary |
| Patient perceived perception of change in | cards. |
| pain | Measure Global Impression of change as |
| Patient medication satisfaction. | compared with Period 2 baseline (BL2) |
| Safety of basimglurant once daily dosing | Patient reported rating of the Medication |
| 1.5-3.5 mg compared with placebo. | Satisfaction Questionnaire (MSQ) |
| | Incidence and severity of adverse events. |
| | Laboratory and cardiovascular safety will also |
| | be evaluated. |

Exploratory

| | |
|---|---|
| The impact of pain on general activities | Recorded ratings of interference of pain |
| of daily living. | with patient's activities captured in |
| | patient diary cards. |

OPEN-LABEL EXTENSION
Primary

| | |
|---|---|
| To evaluate the long-term safety of | Incidence and severity of adverse events. |
| basimglurant daily dosing 1.5-3.5 mg. | Laboratory, vital signs and cardiovascular |
| | safety will also be evaluated. |

Secondary

| | |
|---|---|
| To evaluate the continued efficacy of | Mean scores in the total patient-rated Brief |
| basimglurant with once daily dosing | Pain Inventory-Facial (BPI-F) scale. |
| 1.5-3.5 mg on the following disease aspects: | Frequency and severity of attacks |
| Impact on facial pain | (paroxysms) as well as severity and duration |
| Pain frequency and severity | of continuous pain captured in patient diary |
| Patient perceived severity of pain | cards. |
| | Measure Global Impression of severity as |
| | captured by PGI-S. |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of treating trigeminal neuralgia (TGN), comprising administering to a subject in need thereof a composition comprising a therapeutically effective amount of a therapeutic agent, wherein the therapeutic agent is a compound of Formula I: 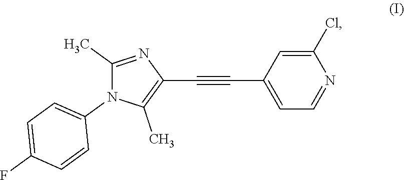 [SMILES: Cc1nc(C#Cc2ccnc(Cl)c2)c(C)n1-c1ccc(F)cc1]
or a pharmaceutically acceptable salt thereof.

2. The method of claim 1, wherein therapeutic efficacy of the treatment is determined by assessing improvement based on the impact on facial pain.

3. The method of claim 1, wherein therapeutic efficacy of the treatment is determined by assessing improvement based on the impact on facial pain, where the subject shows a change from baseline.

4. The method of claim 1, wherein therapeutic efficacy of the treatment is determined by assessing improvement based on the Patient Global Impression of change (PGI-C).

5. The method of claim 1, wherein therapeutic efficacy of the treatment is determined by assessing improvement based on the Patient Global Impression of change (PGI-C), where the subject shows a change from baseline.

6. The method of claim 1, wherein therapeutic efficacy of the treatment is determined by assessing improvement based on the Sheehan Disability Scale (SDS).

7. The method of claim 1, wherein therapeutic efficacy of the treatment is determined by assessing improvement based on the Sheehan Disability Scale (SDS), where the subject shows a change from baseline.

8. The method of claim 1, wherein therapeutic efficacy of the treatment is determined by assessing improvement based on the patient global impression-severity (PGI-S).

9. The method of claim 1, wherein therapeutic efficacy of the treatment is determined by assessing improvement based on the patient global impression-severity (PGI-S), where the subject shows a change from baseline.

10. The method of claim 1, wherein therapeutic efficacy of the treatment is determined by assessing improvement based on the number and severity of pain attacks (paroxysms) as well as duration and severity of continuous pain, and pain interference with daily activities.

11. The method of claim 1, wherein therapeutic efficacy of the treatment is determined by assessing improvement based on the number and severity of pain attacks (paroxysms) as well as duration and severity of continuous pain, and pain interference with daily activities, where the subject shows a change from baseline.

12. The method of claim 1, wherein therapeutic efficacy of the treatment is determined by assessing improvement based on the number of pain free days.

13. The method of claim 1, wherein therapeutic efficacy of the treatment is determined by assessing improvement based on the patient reported rating of the medication satisfaction questionnaire (MSQ).

14. The method of claim 1, wherein the therapeutic efficacy of the treatment is determined by the continued efficacy of the compound of Formula I.

15. The method of claim 1, wherein therapeutic efficacy of the treatment is determined by assessing improvement based on the incidence and severity of adverse event (AE).

16. The method of claim 1, wherein the therapeutic is in its free base form.

17. The method of claim 1, wherein the therapeutic is a pharmaceutically acceptable salt.

18. The method of claim 1, wherein the pharmaceutically acceptable salt is a monosulfate salt or a hemisulfate salt.

19. The method of claim 1, wherein administering of the therapeutic comprises administering the therapeutic in an amount of about 0.1 mg to about 5.0 mg once daily.

20. The method of claim 1, wherein administering of the therapeutic comprises administering the therapeutic in an amount of about 1.0 mg to about 4.0 mg once daily.

21. The method of claim 1, wherein administering of the therapeutic comprises administering the therapeutic in an amount of about 0.1 mg to about 3.5 mg once daily.

22. The method of claim 1, wherein administering of the therapeutic comprises administering the therapeutic in an amount of about 1.5 mg to about 3.5 mg once daily.

23. The method of claim 1, wherein administering of the therapeutic comprises administering the therapeutic in an amount of about 4.0 mg, about 3.5 mg, about 3.0 mg, about 2.5 mg, about 2.0 mg, about 1.5 mg, about 1.0 mg, or about 0.5 mg once daily.

24. The method of claim 1, wherein the therapeutic is administered orally.

25. The method of claim 1, wherein the therapeutic is administered as a unit dose.
